(19) United States
(12) Patent Application Publication
(10) Pub. No.: US 2009/0036705 A1
(43) Pub. Date: Feb. 5, 2009

(54) Production of Alkyl Esters from High Fatty Acid Feedstocks

(75) Inventor: William Douglas Morgan, Richmond, CA (US)

Correspondence Address: KING & SPALDING, LLP, 1100 LOUISIANA ST., STE. 4000, ATTN.: IP Docketing, HOUSTON, TX, 77002-5213, US

(73) Assignee: ENDICOTT BIOFUELS II, LLC, Houston, TX (US)

(21) Appl. No.: 12/172,717

(22) Filed: Jul. 14, 2008

Related U.S. Application Data
(60) Provisional application No. 60/962,689, filed on Jul. 31, 2007.

(51) Int. Cl. C07C 69/003

(52) U.S. Cl. 560/129

(57) ABSTRACT

The present invention relates to a process for the production of fatty acid esters from high fatty acid feedstocks such as vegetable oil soapstocks. Specifically, the present invention relates to the production of ASTM, EN, and IRS specification Biodiesel by saponification and acidulation of soapstock followed by heterogeneous reactive distillation esterification.

This application claims priority under 35 U.S.C. 119(e) to U.S. provisional application 60/962,689, filed Jul. 31, 2007, the contents of which are incorporated by reference in their entirety.

### FIELD OF INVENTION

The present invention relates to a process for the production of fatty acid esters from high fatty acid feedstocks such as vegetable oil soapstocks. Specifically, the present invention relates to the production of ASTM, EN, and IRS specification Biodiesel by saponification and acidulation of soapstock followed by heterogeneous reactive distillation esterification.

### BACKGROUND

Diesel fuel is a refined petroleum product which is burned in the engines powering many of the world's trains, ships, and large trucks. Petroleum is a non-renewable resource of finite supply. Acute shortages and dramatic price increases in petroleum and the refined products derived from petroleum have been experienced by industrialized countries during the past quarter-century. Furthermore, diesel engines which run on petroleum-based diesel emit relatively high levels of certain pollutants, especially particulates. Accordingly, research effort is being directed toward replacing some or all petroleum-based diesel fuel with a cleaner-burning fuel derived from a renewable source such as farm crops, waste animal fats and other suitable waste materials.

In an effort to partially replace dependence on petroleum-based diesel, vegetable oils have been directly added to diesel fuel. These vegetable oils are composed mainly of triglycerides, and often contain small amounts (typically between 1 and 10% by weight) of free fatty acids. Some vegetable oils may also contain small amounts (typically less than a few percent by weight) of mono- and di-glycerides.

Triglycerides are esters of glycerol, CH₂(OH)CH(OH)CH₂(OH), and three fatty acids. Fatty acids are, in turn, aliphatic compounds containing 4 to 24 carbon atoms and having a terminal carboxyl group. Diglycerides are esters of glycerol and two fatty acids, and monoglycerides are esters of glycerol and one fatty acid. Naturally-occurring fatty acids, with only minor exceptions, have an even number of carbon atoms and, if any unsaturation is present, the first double bond is generally located between the ninth and tenth carbon atoms. The characteristics of the triglyceride are influenced by the nature of their fatty acid residues.

The production of alkyl esters of glycerides by transesterification is a known process. However, transesterification suffers in that the reaction generally requires the addition of an acid or base catalyst which must be subsequently neutralized after the reaction thereby generating salts and soaps. In addition, while transesterification results in the separation of fatty acid esters from triglycerides, it also results in the production of glycerin, which must then be separated from the fatty acid esters, glycerin, excess alcohol, salts, and soaps. Furthermore, the use a strong acid, such as sulfuric acid, typically leads to higher sulfur content in the resulting Biodiesel as the acid reacts with the double bonds in the fatty acid chains.

Alkyl esters can also be produced from fatty acids by esterification using acid catalysts such as sulfuric acid, hydrochloric acid, and toluene sulfonic acid. This technology suffers from equilibrium constraints on conversion due to produced water as well as contamination of the product with sulfur. As with wet chemical transesterification, soap is produced as a result of subsequent neutralization. Therefore, wet chemical esterification of fatty acids suffers from similar separation problems associated with co-produced alcohol, water, soaps, and esters.

In an effort to overcome some of the problems associated with transesterification, attempts have been made to employ esterification between fatty acids and alcohols. In these processes fatty acids are prepared from triglycerides by hydrolysis, followed by catalyzed esterification of the fatty acids with an alcohol, preferably methanol. While this practice is practiced in the production of fatty alcohols and fatty acid esters, as described in U.S. Pat. No. 5,536,856 (Harrison et al), it has not been practiced in the production of Biodiesel.

### SUMMARY

The present invention provides a process for the economical production of fatty acid esters suitable for use as Biodiesel under US and European tax and technical standards and specifications from feedstocks containing high levels of free fatty acids. The invention overcomes the prior art that has caused certain feedstocks to be overlooked as economic means of making specification Biodiesel.

In one embodiment, a key feature of the present invention is that it not only facilitates moving high free fatty acid feedstocks into Biodiesel markets, but does so by making use of existing soapstock acidulation technology and capital equipment.

According to one aspect of the invention, raw feedstock comprising or consisting essentially of vegetable soap stocks, and/or other plant or animal derived lipids, is fed to a tank reactor and reacted with NaOH or KOH. The feedstock is treated with sufficient NaOH or KOH, live steam, and hold time to affect complete or near complete splitting of any remaining glycerides into free fatty acids. Preferred levels of pH, temperature, and hold time are discussed in U.S. Pat. No. 6,855,838 and are herein incorporated by reference. After sufficient hold time at sufficient pH and temperature, the mixture in the same or subsequent reactor is treated with sufficient sulfuric acid, additional live steam, and additional hold time to affect “breaking of the soaps” and separation of the mixture into three distinct liquid layers upon settling. The top layer contains fatty acids, also known as high-acid oil (HA). There is next a “rag” layer that contains a mixture of organics and water, including fatty acids. The bottom layer contains water, some organics, and the sodium and/or potassium sulfate that results from the reaction of sulfuric acid and NaOH and/or KOH.

In one aspect of the invention, the rag layer is separated from the other layers and sold “as is” to a downstream user. In another aspect of the invention, the rag layer is subjected to a further round of saponification and acidulation to yield additional fatty acids. The bottom layer is discarded as waste water or partially recycled to the next round of reactions.

In one embodiment of the invention, a top layer, or HA layer, is suitable for use directly as feedstock for the esterification process of the current invention. In another embodiment, the HA layer contains contaminants that should be removed prior to esterification. Such impurities can include sulfur, phosphorous, and metals such as iron. Removal of these impurities can be affected by the appropriate adsorption, distillation, and/or extraction process known to those skilled in these arts. In one embodiment, esterification may be more fully described as heterogeneous reactive distillation esterification.

In another aspect of the invention, the further purified or raw HA layer is fed as fatty acid feedstock to a heterogeneous reactive distillation process, such as described in U.S. Pat. No. 5,536,856 herein incorporated by reference. The HA is converted to alkyl esters by reaction with a suitable alcohol, such as described in U.S. Pat. No. 5,536,856. Suitable alcohols may contain 1-24 carbon atoms. Preferably, the alcohol forming the alkyl group is a C1-C6 alcohol. In one embodiment, the alcohol is selected from methanol, ethanol, propanol, and butanol. In one embodiment the alkyl ester is a methyl ester. If necessary, the crude alkyl ester product is further purified by stripping, distillation, and/or adsorption according to methods known to those skilled in those arts.

The esterification and purification processes are operated such that the resulting alkyl ester product meets US and European Biodiesel specifications. By choice of feedstock, the resulting product can be made to meet additional specifications in addition to Biodiesel. In another embodiment, the processes are performed on an industrial scale.

### BRIEF DESCRIPTION OF THE DRAWINGS

FIG. 1 shows one embodiment of the invention in terms of a sequence of steps.

FIG. 2 shows another embodiment of the invention in terms of a similar sequence of steps with additional purification steps between acidulation and esterification.

FIG. 3 shows another embodiment of the invention in terms of a similar sequence of steps with an additional purification step after esterification.

FIG. 4 shows another embodiment of the invention in terms of a similar sequence of steps with additional purification steps after acidulation and esterification.

### DETAILED DESCRIPTION OF THE INVENTION

The present invention provides a process that combines saponification, acidulation, and heterogeneous reactive distillation to produce alkyl esters from high fatty acid content lipid feedstocks derived from animal and/or vegetable sources. In the present invention, shortcomings found in the current art with regard to water of reaction, equilibrium effects, and sulfur limits are eliminated by employing reactive distillation over a solid catalyst.

In the production of Biodiesel from fatty acids and glycerides, it is extremely advantageous to utilize feedstocks high in free fatty acids due to their lower cost. However, the presence of free fatty acids complicates the production of Biodiesel. Esterification is an acid catalyzed process and transesterification is normally a base catalyzed process. Therefore, it is advantageous to feed high purity glycerides with low fatty acid content to transesterification processes and high purity fatty acids with low glyceride content to esterification processes. For example, vegetable soapstock (SS) is a material that typically contains around 50% free fatty acids and 50% glycerides. Therefore, it poses a quandary in terms of the choice of catalyst. For example, one option is to separate the fatty acids and glycerides by distillation and perform base catalyzed esterification on the glyceride fraction and acid catalyzed esterification on the fatty acid fraction. Another option is to perform acid catalyzed transesterification and esterification at the same time on the whole feedstock. While possible, neither of these approaches avoids complications associated with sulfur contamination, reaction water equilibrium effects, nor post-reaction separation issues.

Vegetable soapstock poses further issues due to its content of water, acylglycerols (glycerides), phosphoacylglycerols, and sodium or potassium soaps of fatty acids. Of these materials, the most serious impediments for making specification Biodiesel are the Na or K ions. Normally, before it is used for its normal purposes (e.g., as animal feed), soapstock's soaps are “broken” via acidulation. Acidulation involves lowering a batch of soapstock's pH by adding sulfuric acid and raising the reaction temperature for sufficient time to cause the Na or K ions to go back into the water phase.

U.S. Pat. No. 6,855,838 discloses a method by which the glycerides in high fatty acid containing lipid mixtures such as soapstock are converted to fatty acids by saponification followed by acidulation to break the soaps. Soapstock, which typically contains a mixture of 50% of fatty acids and 50% triglycerides, is first converted to fatty acids by reaction with a base such as NaOH or KOH prior to breaking of the soaps via acidulation. This process yields a final product which has over 90% free fatty acids and less than 10% glycerides. Because of the nature of the acidulation reaction itself, performing a “pre-saponification” step prior to acidulation does not require any major modifications to the acidulation process. Other high free fatty acid materials that can be fed with soapstock or used in its place include poultry fat, yellow grease, choice white grease, brown grease, feed grade fat, low grade tallow, used restaurant grease, and used grease.

U.S. Pat. No. 6,855,838 also discloses a sulfuric acid catalyzed, wet chemical method for esterification of the “High Acid” Oil (HA) prepared by saponification and acidulation of SS. As disclosed, esterification will not proceed beyond 95% conversion of fatty acids to esters regardless of conditions or reaction time. As further disclosed, the conversion is limited by the equilibrium constraints imposed by reaction water. Two means of achieving higher conversion or otherwise fatty acid free esters may be available. First, washing the remaining fatty acids with a basic water solution may be an option. Second, performing a second esterification reaction after first removing the water of reaction as a lower phase produced by centrifugation may be an option. It is an embodiment of the current invention to avoid equilibrium constraints by performing esterification on HA using heterogeneous reactive distillation where the water of reaction is continuously removed as a vapor distillate in a reaction column.

The present invention involves esterification of fatty acids using heterogeneous reactive distillation and the preferred feedstocks may have fatty acid contents above 90%. Where the art disclosed in U.S. Pat. No. 6,855,838 offers a method for deriving feedstock from relatively high free fatty acid materials such as SS and HA, it nonetheless falls short in its ability to obtain sufficient conversion of fatty acids to esters per US and European tax and technical specifications. Therefore, another embodiment of the present invention is the combination of the art described in U.S. Pat. No. 6,855,838 with the art described in U.S. Pat. No. 5,536,856, where such material made by the process of U.S. Pat. No. 6,855,838 is fed to the process of U.S. Pat. No. 5,536,856 such that sufficient conversion of fatty acid to esters is obtained directly via a single reaction vessel with one or more distillation stages.

While the methods disclosed herein are applicable to feedstocks containing any amount of free fatty acids, the methods according to the invention are particularly applicable to feedstocks with free fatty acids greater than 15%, more preferably greater than 30%, and more preferably greater than 50%. As the free fatty acid content of a feedstock increases, the methods disclosed herein provide additional economic and technical benefits.

By “consisting essentially of” is meant that components that change the fundamental properties of the composition are not included. For example, in one embodiment, “consisting essentially of” indicates a purity of greater than about 80%, 85%, 90%, 91%, 92%, 93%, 94%, 95%, 96%, 97%, 98%, 99%, or higher, so long as the fundamental properties are not altered.

While the methods disclosed herein are applicable to a variety of vegetable and animal based lipid mixtures, methods according to the invention are particularly applicable to vegetable soapstocks produced via the caustic refining of crude vegetable oils since, in order to utilize these oils for Biodiesel production, the oils would need to first go through a soap removal/acidulation step that, as noted above, can be performed in conjunction with a pre-saponification step that effectively raises the free fatty acid content of the feedstock. In a preferred embodiment, vegetable soapstocks comprise ideal feedstocks for the present invention because the capital infrastructure for performing the saponification and acidulation methods disclosed herein exists and only requires modification according to the present invention. Further, these materials may be available at a discount compared to the refined oils from which they were previously separated. Vegetable soapstocks are produced as the result of chemical refining of the seeds of a variety of oil crops including, but not limited to, soy, cotton, coconut, palm, rapeseed, canola, safflower, corn, canola, sunflower, flax, and jatropha.

In one embodiment, the process is performed on an industrial scale. For example, in a preferred embodiment, production occurs from 500 kg or more of feedstock per day. Alternatively, production may occur on batches of 1,000 kg, 5,000 kg, 10,000 kg or more of feedstock per day. Global biodiesel production is estimated at several million tons per year.

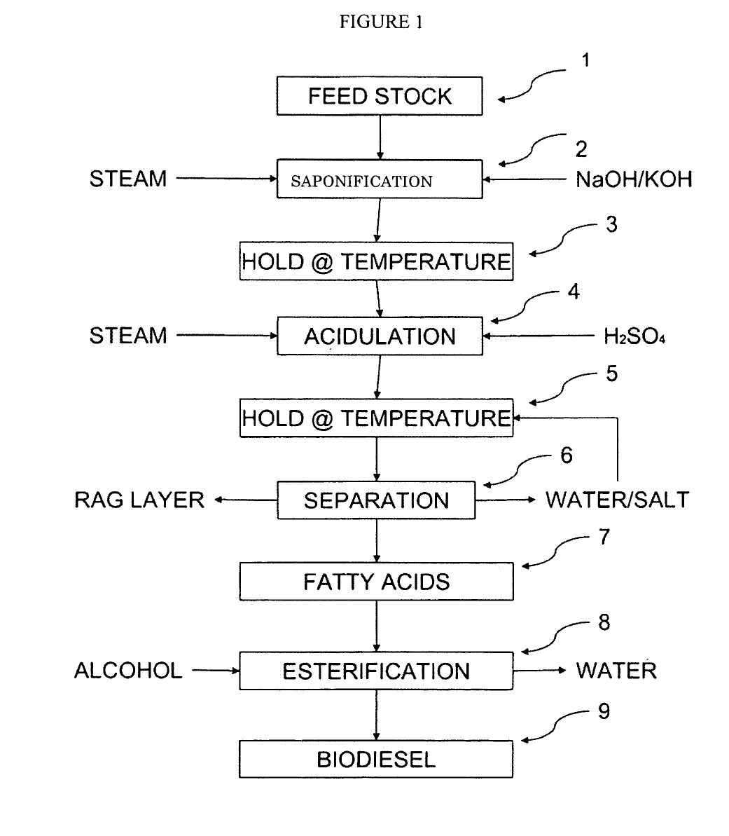

Referring to FIG. 1, feedstock 1 consists of vegetable soapstock and/or other fatty acid containing lipids of vegetable or animal origin. The material can be virgin or recycled. The higher the free fatty acid in the feedstock, the more applicable the process of the current invention since less NaOH or KOH will be required to affect complete saponification. Feedstock 1 is fed to a reactor vessel, 2, to which live steam and NaOH and/or KOH are added. The results of varying the amount of free fatty acid in the feedstock and the amount of NaOH and/or KOH and steam added are discussed in U.S. Pat. No. 6,855,838 which is incorporated by reference.

Once sufficient steam and NaOH have been added, the mixture is held at temperature in step 3. While held, samples can be taken to gauge the degree of saponification, i.e. the ratio of acid number to saponification number. The goal is for this ratio to be greater than 0.80, preferably greater than 0.90, preferably greater than 0.92, preferably greater than 0.96, and if possible, greater than 0.99. The optimum nearness to 1.0 for this ratio will depend on downstream effects that will vary for different feedstocks.

After sufficient hold time, 3, additional steam and sulfuric acid are added to the mixture, step 4, in order to break the soaps that formed as the result of steps 2 and 3. This mixture is then held at temperature in step 5. The amount of steam and sulfuric acid added in step 4 and the amount of hold time utilized in step 5 will depend on the feedstock and other factors as discussed in U.S. Pat. No. 6,855,838 which is incorporated by reference.

After hold step 5, the mixture is allowed to settle and separate, 6. The mixture separates into at least two layers—upper and lower—and more likely three—upper, rag, and lower. The rag layer, if formed, can be removed and sold or further subjected to processing as raw feed, 1, to the process. The lower layer can be discarded as waste water or partially recycled as shown. Recycling of the mixture has the effect of improving the separation step, 6, during subsequent iterations of the process.

In one embodiment of the invention, the upper layer, 7, containing high acid number to saponification value fatty acids is fed as is to one of the various embodiments of the esterification process, 8. As an example, see an embodiment in U.S. Pat. No. 5,536,856, hereby incorporated by reference. The resulting products from the esterification process, 8, are water and specification Biodiesel or other alkyl esters.

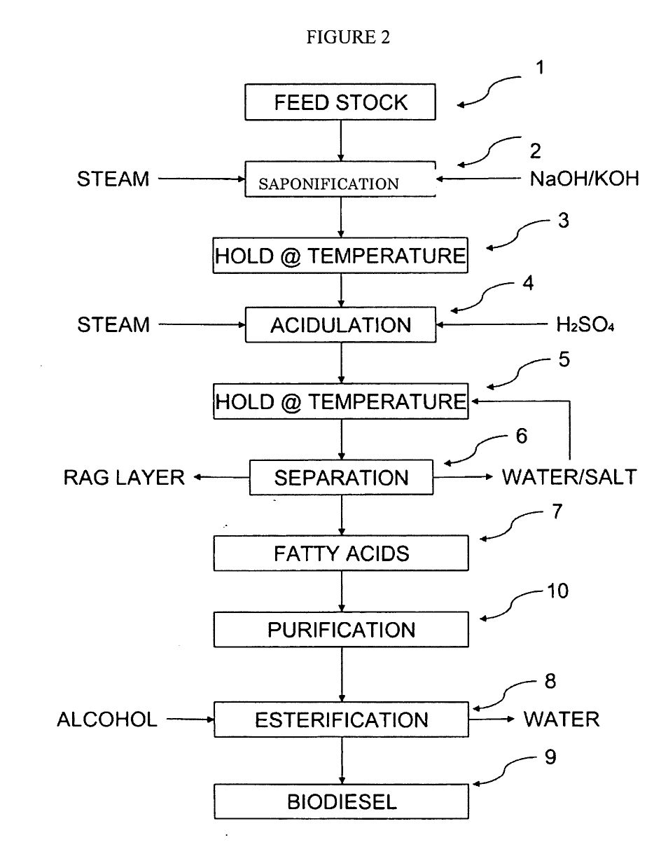

In another embodiment of the invention, FIG. 2, the fatty acid layer, 7, is subjected to a purification step, 10, selected from adsorption, distillation, and/or extraction. The purpose of this step is to remove components that either harm the catalyst in step 8 or which are undesirable in the final Biodiesel product and which are more preferentially removed from fatty acids rather than esters.

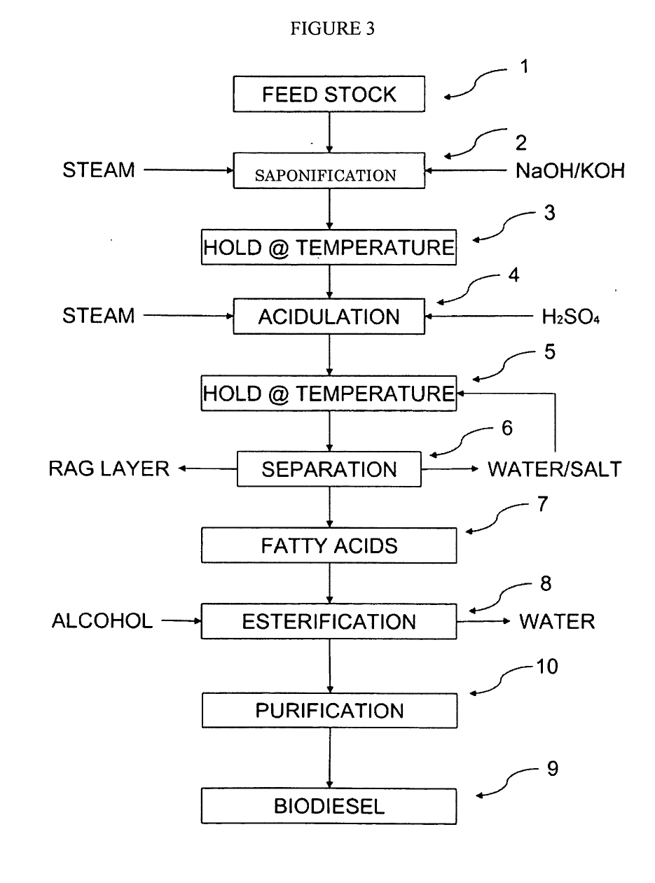

In another embodiment of the invention, FIG. 3, the ester product from esterification, 8, is subjected to a purification step, 10, selected from adsorption, distillation, and/or extraction.

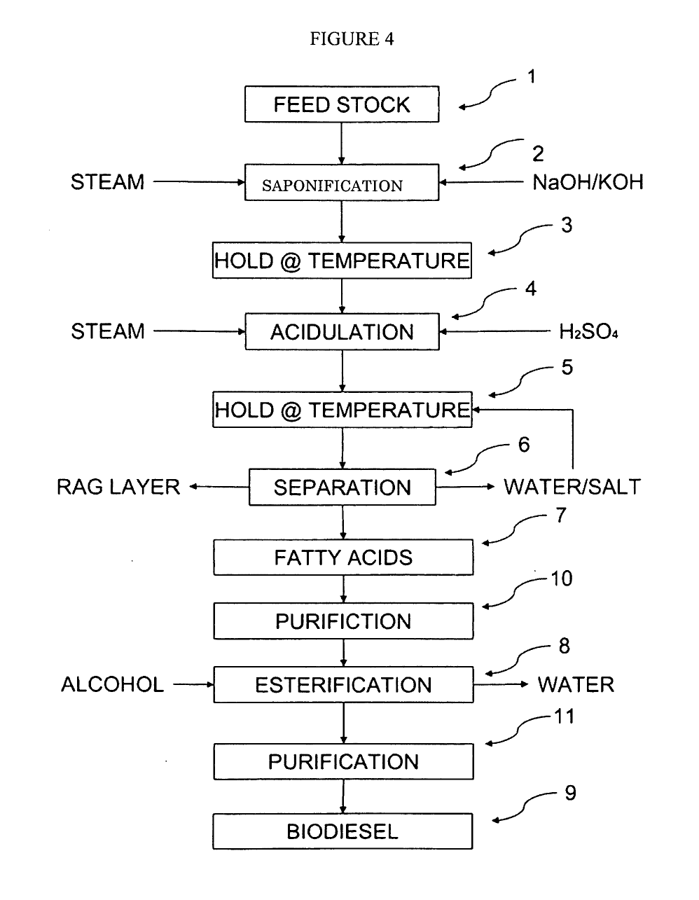

In a further embodiment of the invention, FIG. 4, the fatty acid layer, 7, is subjected to a purification step, 10, selected from adsorption, distillation, and/or extraction and the ester product from the esterification step, 8, is subjected to a purification step, 11, selected from adsorption, distillation, and/or extraction.

It will be understood by those skilled in the art that the drawings are diagrammatic and that further items of equipment such as reflux drums, pumps, vacuum pumps, temperature sensors, pressure sensors, pressure relief valves, control valves, flow controllers, level controllers, holding tanks, storage tanks, and the like may be required in a commercial plant. The provision of such ancillary items of equipment forms no part of the present invention and is in accordance with conventional chemical engineering practice.

Modifications and variations of the present invention relating to the selection of reactors, feedstocks, alcohols and catalysts will be obvious to those skilled in the art from the foregoing detailed description of the invention. Such modifications and variations are intended to come within the scope of the appended claims. All numerical values are understood to be prefaced by the term “about” where appropriate. All references cited herein are hereby incorporated by reference in their entirety.

1. A process for the production of an alkyl ester comprising:
i) obtaining soapstock as feedstock for the process;
ii) performing a saponification step;
iii) performing an acidulation step;
iv) performing a separation step to yield a fatty acid layer; and
v) esterifying the fatty acid layer to yield an alkyl ester product.

2. The process according to claim 1, wherein the process is performed on an industrial scale.

3. The process according to claim 1, wherein the soapstock is vegetable based.

4. The process according to claim 3, wherein the soapstock is the result of chemical refining of the seeds of an oil crop selected from soy, cotton, coconut, palm, rapeseed, canola, safflower, corn, canola, sunflower, flax, jatropha, and mixtures thereof.

5. The process according to claim 1, wherein saponification includes steam and treatment with sodium hydroxide or potassium hydroxide.

6. The process according to claim 1, wherein acidulation includes steam and treatment with H₂SO₄.

7. The process according to claim 1, wherein separation includes removal of a rag layer and an aqueous layer.

8. The process according to claim 1, wherein esterification is heterogeneous reactive distillation esterification.

9. The process according to claim 8, wherein the esterification occurs via a gas sparged, slurry form of heterogeneous reactive distillation in a reaction chamber.

10. The process according to claim 1, wherein the alkyl ester is Biodiesel according to ASTM, EN, and/or IRS specification.

11. The process according to claim 1, wherein the alkyl ester is a methyl, ethyl, propyl, or butyl ester.

12. The process according to claim 1, further comprising purification between separation and esterification steps.

13. The process according to claim 1, further comprising purification following esterification.

14. The process according to claim 1, further comprising purification between separation and esterification steps, and purification following esterification.

15. The process according to claim 1, wherein the feedstock consists essentially of soapstock.

16. The process according to claim 1, wherein glycerin, soap, alcohol, and water contents in the alkyl ester product are less than 0.5% by weight.
